(19) United States
(12) Patent Application Publication
(10) Pub. No.: US 2023/0100722 A1
(43) Pub. Date: Mar. 30, 2023

(54) MECHANOCHEMICAL SNAr REACTIONS

(71) Applicant: University of Cincinnati, Cincinnati, OH (US)

(72) Inventor: Joel Andersen, Cincinnati, OH (US)

(21) Appl. No.: 17/794,003

(22) PCT Filed: Dec. 7, 2020
(86) PCT No.: PCT/US20/63601

§ 371 Date: Jul. 20, 2022

Related U.S. Application Data
(60) Provisional application No. 62/944,803, filed on Dec. 6, 2019.

(51) Int. Cl. C07D 295/06; C07C 201/12; C07C 319/14

(52) U.S. Cl. CPC C07D 295/06; C07C 201/12; C07C 319/14

(57) ABSTRACT

The present invention involves a mechanochemical process in which at least two reactants are mixed without an additional solvent to produce an SNAr reaction product. In one embodiment, the mixing is achieved using a twin-screw extruder. In another embodiment, the mixing is achieved using dry mixing equipment. In one embodiment, the dry mixing equipment is selected from the group consisting of batch Paddle Mills, continuous Paddle Mills, V-Blenders, Twin Cone Blenders and Ribbon Blenders. In another embodiment, the mixing is achieved using a Fluidized Bed reactor.

### CROSS REFERENCE TO RELATED APPLICATIONS

This application claims priority to U.S. Provisional Application Ser. No. 62/944,803, filed Dec. 6, 2019, which application is hereby incorporated by reference in its entirety.

### TECHNICAL FIELD

The present invention relates mechanochemical reactions.

### BACKGROUND OF THE INVENTION

Nucleophilic aromatic substitution (S_{N}Ar) reactions have received attention as a valuable target for developing environmentally friendly reaction conditions. Recent investigations have ranged from micelle catalysis to ionic liquids to alternative solvents such as Cyrene. The value of this target derives from the popularity of S_{N}Ar reactions in pharmaceuticals and agrochemicals despite their reliance on polar, aprotic solvents. The ACS Green Chemistry Institute Pharmaceutical Roundtable, a collection of engineers and scientists from industry interested in addressing the need for sustainability, included finding alternatives for these solvents in their list of top 12 priorities. These solvents can be troublesome, and potentially costly for manufacturing use due to their high toxicity and tedious removal during work-up and contaminated aqueous waste streams resulting from their water miscibility and high boiling points. These concerns have led to potential strict regulation in the future under European Union REACH legislation. This has led to seeking alternatives for such solvents. However, the very characteristics that make these solvents desirable for reaction purposes (polar and high boiling point) can frustrate the process of identifying a simple “drop-in” solvent capable of replacing undesirable polar, aprotic solvents in a robust manner.

### SUMMARY OF THE INVENTION

The present invention involves a mechanochemical process in which at least two reactants are mixed without an additional solvent to produce an SNAr reaction product. In one embodiment, the at least two reactants are mixed at a constant temperature. In another embodiment, the constant temperature is in the range of −10° C. to 100° C. In one embodiment, the constant temperature is in the range of 25° C. to 80° C. In another embodiment, at least about 80% of the products of the reaction are the SNAr reaction product. In one embodiment, at least about 90% of the products of the reaction are the SNAr reaction product.

In one embodiment, the mixing is achieved using a twin screw extruder. In another embodiment, the mixing is achieved using dry mixing equipment. In one embodiment, the dry mixing equipment is selected from the group consisting of batch Paddle Mills, continuous Paddle Mills, V-Blenders, Twin Cone Blenders and Ribbon Blenders. In another embodiment, the mixing is achieved using a Fluidized Bed reactor.

### BRIEF DESCRIPTION OF THE DRAWINGS

FIG. 1 is a pair of graphs showing kinetic studies of the reaction under conventional conditions at high concentrations (0.5 M and 1.0 M) in DMF, as well as under mechanochemical conditions at the same temperature. FIG. 1A shows results based on conversion percentage and FIG. 1B shows results based on mole fraction.

FIG. 2 is a graph showing the data from FIG. 1A.

FIG. 3 is a graph showing that changing the leaving group from chlorine to fluorine results in drastically increased kinetics.

FIG. 4 is a graph showing that replacing the nucleophile with less reactive benzyl amine resulted in drastically slower kinetics.

FIG. 5 is a graph showing the results of changing to an alcohol nucleophile and a different aromatic system.

FIG. 6 is a graph showing that a thiol reacted very favorably with an ortho-substituted ring under mechanochemical conditions

FIG. 7 is a graph showing the temperature-dependence of the reaction conversion when performed in a twin-screw extruder.

### DETAILED DESCRIPTION OF THE INVENTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “Nucleophilic Aromatic Substitution (SNAr)” means substitution of a leaving group for a nucleophile on an aromatic ring. Typically, the ring will be electron-deficient. Broadly speaking, this can be achieved through the presence of a) a single strongly electron withdrawing group, such as —NO2, b) several more weakly electron-withdrawing groups, such as —Cl, or c) an aromatic ring itself containing heteroatoms that is sufficiently activated towards such reactivity.

The following formulae are examples of moieties that can activate an aromatic ring towards nucleophilic aromatic substitution. LG refers to leaving group.

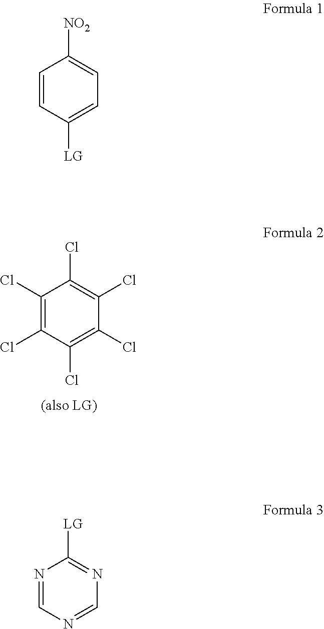

As used herein, the term “solvent” means a chemical present in quantities sufficient to provide bulk dissolution for some or all starting materials, intermediates, and products.

As used herein, the term “mechanochemical process” means a group of reactants, reagents, intermediates, and products undergoing a chemical transformation in an automated mixer in the absence of solvent.

As used herein, the term “liquid-assisted grinding” means stochiometric or sub-stoichiometric amounts of a liquid whose role is to increase ease of mixing or otherwise affect the reaction of an SNAr reaction occurring in a mechanochemical system.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Consider at this point, mechanochemical reactions. These solvent-free reactions use grinding/crushing/pulverizing/shearing forces to induce chemical reactivity. On small scale they are performed in vibrational or planetary ball mill reactors, and on large scale they have seen significant success in twin-screw extruders. Of interest here is that in mechanochemical reactions there is no need for dissolving power and without solvent there are reduced practical limits on reaction temperature. Now that strict control of temperature in vibratory ball mill reactors has been demonstrated several times in literature, the door is open to applying mechanochemistry to reactions like the S_{N}Ar.

A possible benefit of applying mechanochemistry to SNAr reactions is the potential for rate enhancements. These are regularly observed in mechanochemical conditions. Such enhancements would be especially useful for SNAr reactions, as high temperatures and long reaction times are typical. The underlying cause of the enhancements is generally believed to originate from the comparatively high “concentrations” of the starting materials. However, given that temperature control of mechanochemical reactors has only recently emerged, clearer comparisons of solution versus mechanochemistry would be beneficial. Studying the SNAr reaction under a variety of conditions is expected to provide further insight on this.

The present invention is a novel process for a robust, highly effective solvent-free approach to nucleophilic aromatic substitution reactions (S_{N}Ar) achieved through automated mechanical mixing. Conventionally, these reactions are often slow and require high temperatures while also typically relying on polar, aprotic solvents. Advantages of the solvent-free approach include rate, selectivity, and yield enhancements, as well as a simplified work-up resulting a cleaner aqueous waste stream due to the avoidance of polar, aprotic solvents, which are generally miscible with water. The present invention involves a mechanochemical process in which at least two reactants are mixed without an additional solvent to produce an SNAr reaction product. In one embodiment, the at least two reactants are mixed at a constant temperature. In another embodiment, the constant temperature is in the range of −10° C. to 100° C. In one embodiment, the constant temperature is in the range of 25° C. to 80° C. In another embodiment, at least about 80% of the products of the reaction are the SNAr reaction product. In one embodiment, at least about 90% of the products of the reaction are the SNAr reaction product.

In one embodiment, the mixing is achieved using a twin screw extruder. In another embodiment, the mixing is achieved using dry mixing equipment. In one embodiment, the dry mixing equipment is selected from the group consisting of batch Paddle Mills, continuous Paddle Mills, V-Blenders, Twin Cone Blenders and Ribbon Blenders. In another embodiment, the mixing is achieved using a Fluidized Bed reactor.

In the examples of the present invention, reaction rate enhancements were observed. The average rate enhancement for the reactions in Examples 1-4 was 9.0×. These enhancements open the door to the use of cheaper, generally more commercially available starting materials with chlorines instead of fluorines as leaving groups. Conventionally, the use of solvents other than polar, aprotic ones can lead to drastic increases in reaction times. Proper stabilization of the transition state without overstabilization of the starting materials is an important factor in these reactions. Thus, the magnitude of the rate is surprising given the attention provided to solvent selection when identifying reaction conditions. In a conventional S_{N}Ar reaction, the solvent has the highest mole fraction of all chemical components and is generally unchanged during the reaction. However, in a solvent-free mechanochemical reaction, the mole fractions of starting materials, intermediates, and products sum to 1 and are in flux throughout the reaction. Thus, the chemical environment is constantly changing, and it was unclear how that would affect the feasibility of robustly performing S_{N}Ar reactions.

We began investigations with the reaction between 1-chloro-4-nitrobenzene and piperidine, as outlined in Scheme 1.

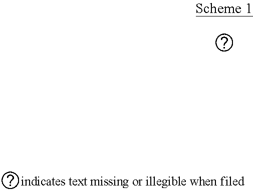

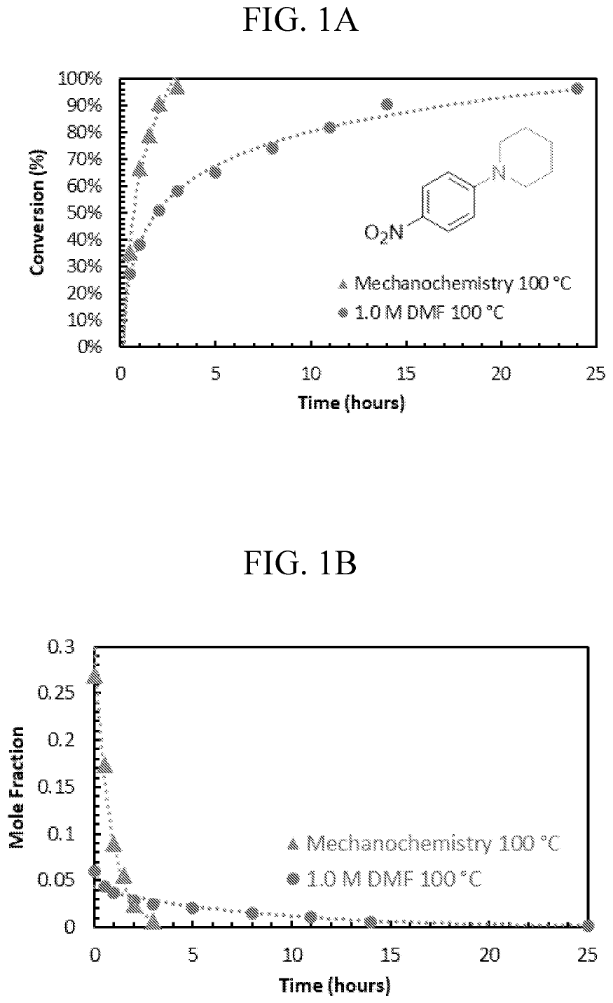

The corresponding product was an intermediate in the synthesis of biologically active drug candidates in a study by Chu-Farseeva et al., and it is representative of a typical SNAr. Furthermore, it is convenient for initial study as no side products are observed under typical reaction conditions. This is despite the fact that, like other SNAr reactions involving only moderately electron-deficient rings, high temperatures and long reaction times are characteristic for this pair of reactants to achieve usable yields. In the original work, the reaction was performed at 100° C. and required 16+ hours to reach 90%+ yields. To provide a head-to-head comparison, we performed kinetic studies of the reaction under conventional conditions at high concentrations (0.5 M and 1.0 M) in DMF, as well as under mechanochemical conditions at the same temperature. The results of these comparisons are presented in FIG. 1A. Increasing the concentration beyond 1.0 M resulted in an inhibitory effect, possibly due to poor mixing. The clear rate advantage of mechanochemical conditions was very encouraging. There are several potential explanations for this rate enhancement, and all are likely involved. First, the solvent-free conditions in a mill result in highly concentrated conditions, which would be expected to provide rate enhancements so long as the mechanochemical reactor maintains perfectly mixed conditions. Second, the lack of solvation means nucleophiles receive no solvent stabilization. As a parallel, it is well established in SNAr literature that protic solvents are generally avoided because although these solvents solubilize reactants, protic solvents overreach and inhibit nucleophilic character via hydrogen-bonding, especially when the nucleophile is relatively small. Hence, aprotic solvents are used to keep the nucleophiles dissolved, yet relatively unencumbered. Since there is no solvation needed under mechanochemical conditions, nucleophiles are left unhindered. However, the initial mole fractions of starting materials and products are so high in mechanochemical systems (in the present reaction they sum to 1), it must be considered that reactants/reagents, products, and intermediates—whose relative amounts are all in flux—control the chemical environment. To this end, FIG. 1B provides some insight into the mole fraction of 1-chloro-4-nitrobenzene during the course of each method. Mole fraction helps to normalize solution concentrations to mechanochemical “concentrations.” Note that at the two-hour mark, the mechanochemical reaction has progressed sufficiently such that the starting material becomes more dilute than in the solution reaction. This is despite the presence of solvent. However, it should be noted that the reaction rate continues to exceed the solvent-based method even though their mole fractions are comparable after this point. If the rate enhancement was merely coming from high “concentrations,” then the rates would be expected to coalesce.

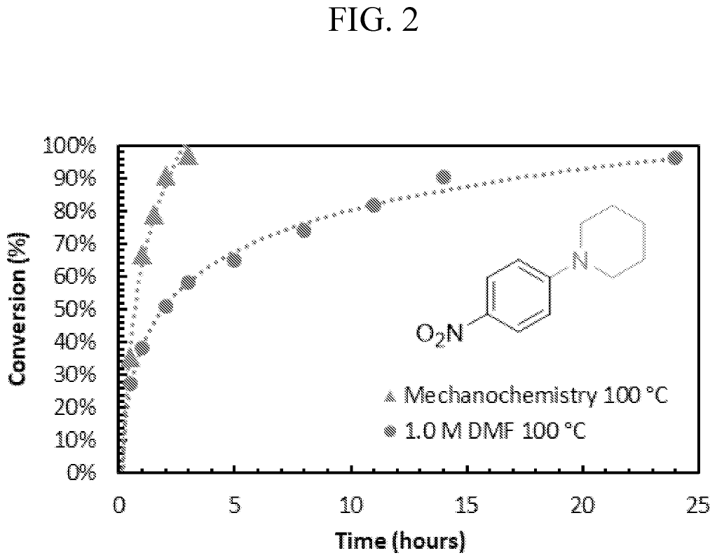

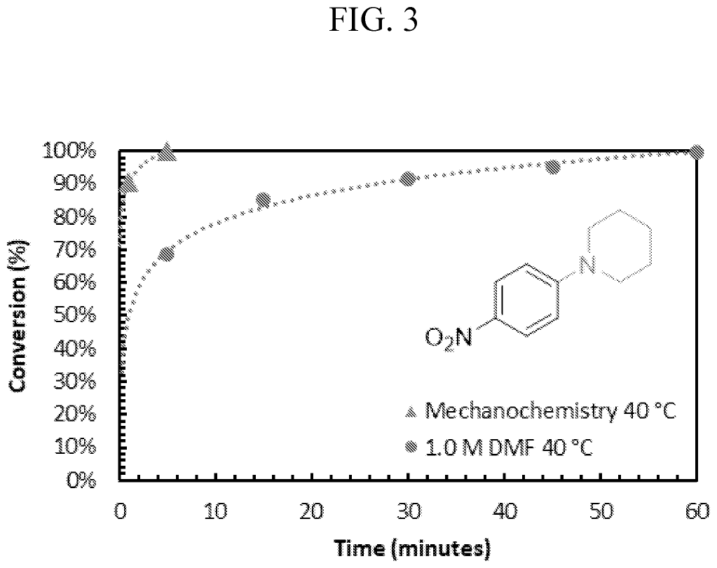

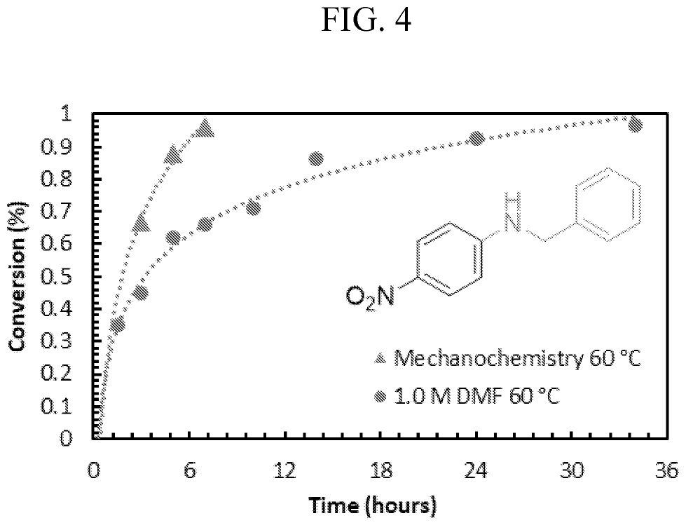

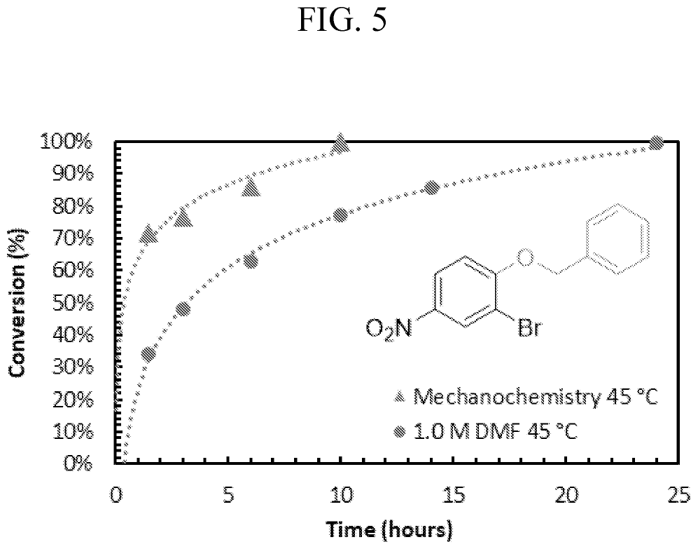

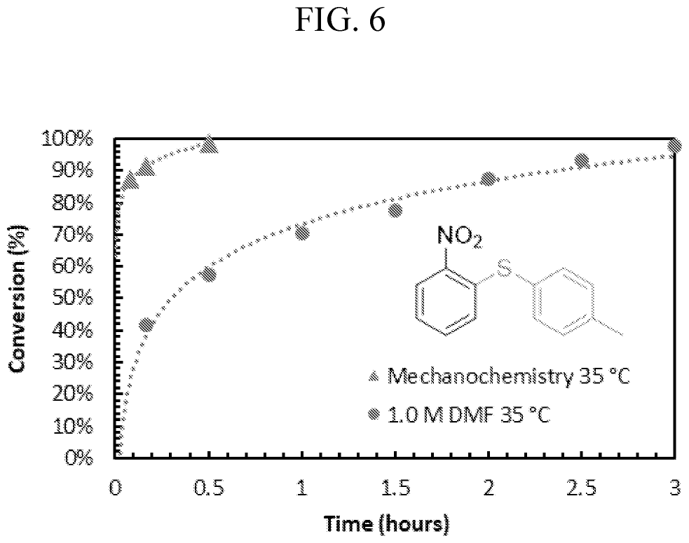

To see if this rate enhancement extended beyond the first set of reactants, various nucleophile and electrophile pairs were studied. The results of these kinetic experiments are presented in FIGS. 2-6. For the sake of comparison, results of FIG. 1(a) are incorporated as FIG. 2. FIG. 3 indicates that changing the leaving group from chlorine to fluorine results in drastically increased kinetics, which is consistent with conventional S_{N}Ar reactions. Pleasingly, the mechanochemical rate enhancement was also observed in this case, and the reactor did not appear to struggle to sufficiently maintain proper mixing despite the short reaction time. Replacing the nucleophile with less reactive benzyl amine in FIG. 4 resulted in drastically slower kinetics, as expected, for both solution and mechanochemical conditions. However, enhanced rates were still observed mechanochemically in comparison to solution. Changing to an alcohol nucleophile and a different aromatic system, FIG. 5, once again indicated that mechanochemical conditions offered a competitive edge from a rate perspective. Similarly, a thiol reacted very favorably with an ortho¬-substituted ring (FIG. 6) under mechanochemical conditions in comparison to solution.

Further results of these studies are summarized in Table 1. In this table, a comparison between milling and solution approaches is provided on the basis of the time required to reach 95% conversion. On average, mechanochemical reactions were [9×] faster than corresponding solvent-based reactions. As a corollary, this increases the practicality of chlorine as a leaving group. Although fluorine offers convenience as the faster leaving group, such fluorinated compounds can be expensive in comparison to chlorinated counterparts. Beyond rate enhancements, isolated yields were, in all cases, higher when mechanochemistry was used. Also of interest is that aqueous waste streams are not contaminated with water-miscible and highly toxic polar, aprotic solvents. In many mechanochemical cases, a simple water wash could suffice to purify the product, making organic solvents unnecessary.

TABLE 1

| | | Mill | Solution |
|---|---|---|---|
| Reaction | | Isolated | Isolated |
| Yield (%) | t_{Sol'n}/t_{Mill} | Yield (%) | Yield (%)^{b} |
| A | 9.0 | 99 | 94 |
| B | 20 | 96 | 92 |
| C | 4.1 | 96 | 86 |
| D | 2.4 | 85 | 81 |
| E | 9.3 | 94 | 83 |
| | Average: 9.0 | | |

In the above cases, there was not much opportunity for side reactions, barring hydrolysis of the electron-deficient aromatic rings. However, the ability to control undesirable reactions is essential to a robust methodology. To investigate the ability of mechanochemistry to control selectivity, a starting material containing both a primary alcohol and primary amine was selected to react with an aromatic system and is outlined in Scheme 2.

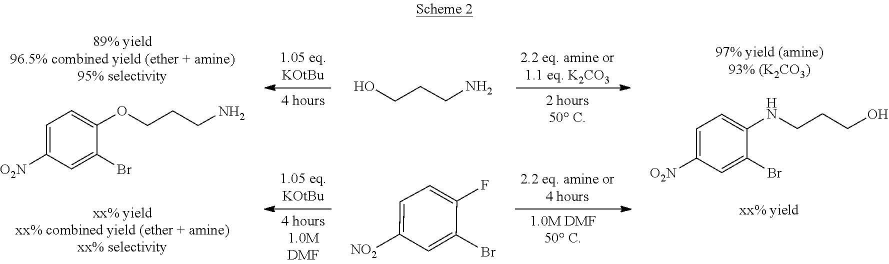

Complete selectivity and excellent yields for amination could be obtained by using either excess amine or a weak base such as K2CO3. To obtain an aryl ether from a pair of reactants like this, sodium hydride would typically be used, although there are safety considerations to be made when using sodium hydride in DMF. In mechanochemical conditions, the relatively safer base potassium tert-butoxide was able to provide excellent selectivity (95% by 1H-NMR) for etherification over amination, granting the ether product in 89% yield. Attempts to further increase selectivity by increasing the equivalents of base resulted in a deep-red, poorly soluble side product, possibly from subsequent nucleophilic action by the amine.

Mixing the reactants may be achieved using a variety of dry mixing equipment, including twin screw extruders (TSE), Paddle Mills (batch or continuous), V-Blenders (e.g., vibratory ball mill), Twin Cone Blender and Ribbon Blenders. Fluidized Bed reactors may also be used.

In conclusion, S_{N}Ar reactions have been carried out in the absence of solvent to great effect. Advantages include rate selectivity, and yield enhancements, as well as a simplified work-up resulting a cleaner aqueous waste stream due to the avoidance of polar, aprotic solvents.

### EXAMPLES

The present mechanochemical reactions were performed in a vibratory ball mill that had been modified in a manner that allowed temperature control over the reaction. Reactivity of rings towards S_{N}Ar reactions is achieved by using a polyhalogenated starting material, the presence of a nitro (—NO₂) group (see Example 1), or the presence of heteroatoms in the ring, preferably by the presence of a nitro group (Example 1) or the presence of heteroatoms in the ring. The leaving groups may be nitro, chloro, or fluoro groups, preferably chloro (Example 1) or fluoro (see Example 2) groups. To neutralize the formation of H⁺ or to increase nucleophilicity of nucleophiles by deprotonation, a variety of bases may be used such as potassium carbonate, potassium phosphate tribasic, potassium phosphate tribasic monohydrate, potassium tertbutoxide, triethylamine, diisopropylethylamine, preferably an inorganic base (Examples 1-3). Nucleophiles may come from salts such as KF or KCN, or, preferably, from neutral sources such as amines (Examples 1-2), alcohols (Example 3), or thiols (Example 4).

### Example 1

1-chloro-4-nitrobenzene (500.0 mg, 1.0 eq), piperidine (405.3 mg, 1.5 eq), and potassium carbonate (526.3 mg, 1.2 eq) were combined in a 15 mL reaction jar. The jar was placed in a modified SPEX-800M Mixer/mill and run for three hours at 100° C. The reaction mixture was extracted from the jar with ethyl acetate and water and transferred to a separatory. After liquid-liquid extraction using additional portions of ethyl acetate, the organic layers were combined, washed with water and brine, dried with sodium sulfate. Ethyl acetate was removed via rotary evaporation. The yield was 99.5% and the purity was >99% by H¹-NMR and GC-MS. The reaction is shown in Scheme 3.

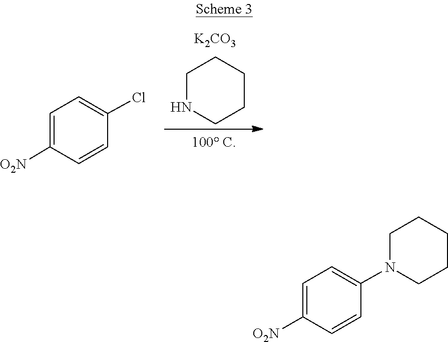 [SMILES: "K2CO3" ; O=[N+]([O-])c1ccc(Cl)cc1 ; C1CCNCC1 ; O=[N+]([O-])c1ccc(N2CCCCC2)cc1]

### Example 2

1-fluoro-4-nitrobenzene (500.0 mg, 1.0 eq), piperidine (452.6 mg, 1.5 eq), and potassium carbonate (587.7 mg, 1.2 eq) were combined in a 15 mL reaction jar. The jar was placed in a modified SPEX-800M Mixer/mill and run for 15 minutes at 40° C. The reaction mixture was extracted from the jar with ethyl acetate and water and transferred to a separatory. After liquid-liquid extraction using additional portions of ethyl acetate, the organic layers were combined, washed with water and brine, dried with sodium sulfate. Ethyl acetate was removed via rotary evaporation. The yield was 96% and the purity was >98% by GC-MS. The reaction is shown in Scheme 4.

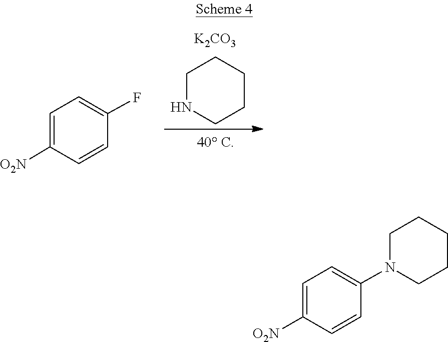 [SMILES: "K2CO3" ; O=[N+]([O-])c1ccc(F)cc1 ; C1CCNCC1 ; O=[N+]([O-])c1ccc(N2CCCCC2)cc1]

### Example 3

2-bromo-1-fluoro-4-nitrobenzene (500.0 mg, 1.0 eq), benzyl alcohol (245.8 mg, 1.0 eq), and potassium carbonate (376.9 mg, 1.2 eq) were combined in a 15 mL reaction jar. The jar was placed in a modified SPEX-800M Mixer/mill and run for ten hours at 45° C. After work-up and column chromatography, the isolated yield of the product was 85%. An equivalent solution experiment produced an isolated yield of 81%. The reaction is shown in Scheme 5.

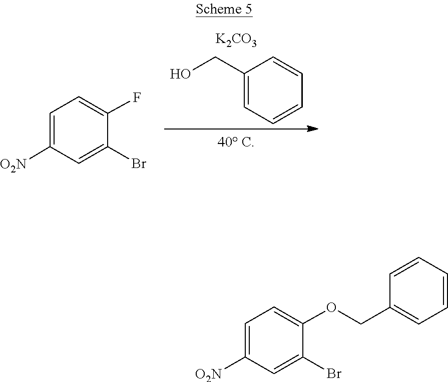

### Example 4

1-fluoro-2-nitrobenzene (500.0 mg, 1.0 eq), 4-methylbenzethiol (440.1 mg, 1.0 eq), and potassium phosphate tribasic monohydrate (816.0 mg, 1.0 eq) were combined in a 15 mL reaction jar. The jar was placed in a modified SPEX-800M Mixer/mill and run for thirty minutes at 35° C. After work-up and column chromatography, the isolated yield of the product was 94%. The reaction is shown in Scheme 6.

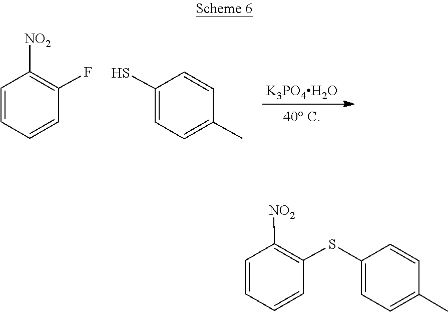

### Example 5

2-bromo-1-fluoro-4-nitrobenzene (200.05 g., 1.0 eq) and potassium carbonate (131.95 g, 1.05 eq) were premixed and added to a twin-screw feeder. This mixture was fed into a ThermoFisher Scientific Process 11 Twin-Screw extruder feedzone at a rate of 1.12 grams/minute. In the following zone, benzyl amine was added via syringe pump at a rate of 0.353 mL/min (1.05 eq). Temperature control over the heating zones was possible via the extruder's software interface. A residence time of about 15 minutes was achieved via the following screw configuration: [D\C×7\A90×8\F60×4\C×7\HF\HR\R60×6\C×19] (D=discharge element; C=forward conveying element; A90×8=alternating 90 degree mixing section consisting of eight one-quarter L:D elements; F60=forwarding 60 mixing; HF=forward conveying, one-half L:D element; HR=reverse conveying, one-half L:D; R60=reverse mixing at 60 degrees). Reaction progress was tracked by high pressure liquid chromatography. Strong control over conversion was demonstrated via varying temperatures of the extruder's various heating zones. Conversions ≥97% were achieved by heating zones 4-8 to 90° C. The reaction is shown in Scheme 7.

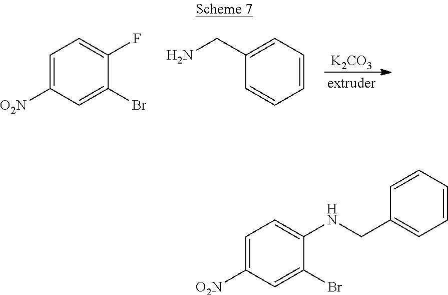

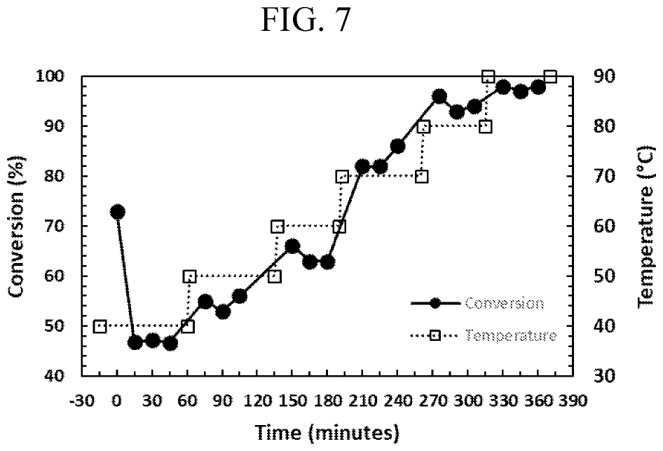

The results of Example 5 are shown in FIG. 7. The graph shows the temperature-dependence of the reaction conversion when performed in a twin-screw extruder.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A mechanochemical process comprising mixing at least two reactants without an additional solvent to produce an S_{N}Ar reaction product.

2. The process of claim 1 wherein the at least two reactants are mixed at a constant temperature.

3. The process of claim 2 wherein the constant temperature is in the range of −10° C. to 100° C.

4. The process of claim 2 wherein the constant temperature is in the range of 25° C. to 80° C.

5. The process of claim 1 wherein at least about 80% of the products of the reaction are the S_{N}Ar reaction product.

6. The process of claim 1 wherein at least about 90% of the products of the reaction are the SNAr reaction product

7. The process of claim 1 wherein mixing is achieved using a twin screw extruder.

8. The process of claim 1 wherein mixing is achieved using dry mixing equipment.

9. The process of claim 8 where the dry mixing equipment is selected from the group consisting of batch Paddle Mills, continuous Paddle Mills, V-Blenders, Twin Cone Blenders and Ribbon Blenders.

10. The process of claim 1 wherein mixing is achieved using a Fluidized Bed reactor.
